(19) United States
(12) Patent Application Publication
(10) Pub. No.: US 2012/0190755 A1
(43) Pub. Date: Jul. 26, 2012

(54) REACTIVE 1-COMPONENT ROADWAY MARKING

(75) Inventors: Mandy Muehlbach, Seligenstadt (DE); Patrick Stenner, Hanau (DE); Silke Suhr, Albstadt-Alzenau (DE); Peter Neugebauer, Limburg (DE); Heike Heeb, Bickenbach (DE); Guenter Schmitt, Darmstadt (DE); Peter Reinhard, Dreieich-Dreieichenhain (DE)

(73) Assignee: EVONIK ROEHM GMBH, Darmstadt (DE)

(21) Appl. No.: 13/499,184

(22) PCT Filed: Sep. 7, 2010
(86) PCT No.: PCT/EP2010/063070

§ 371 Date: Mar. 29, 2012

(30) Foreign Application Priority Data
Oct. 30, 2009 (DE) 10 2009 046 251.1

(51) Int. Cl. C09D 133/10; A61K 8/81; C09J 133/10; A61K 6/00; C08K 3/34; A61K 47/32

(52) U.S. Cl. 514/772.4; 524/853; 523/172; 524/789; 523/105

(57) ABSTRACT

The invention relates to a one-component, storage-stable formulation for marking road surfaces. The invention in particular relates to a formulation for roadway marking comprising encapsulated radical initiators which do not influence the storage stability of the roadway marking and are simple to break open upon application in order to release the initiator.

### FIELD OF THE INVENTION

The present invention comprises a single-component formulation with good shelf life for marking road surfaces. In particular, the present invention comprises a formulation for roadmarking which comprises encapsulated free-radical initiators which do not affect the shelf life of the roadmarking and which are easy, during application, to rupture for release of the initiator.

Single-component reactive systems can be used in a wide variety of sectors. Systems of this type are particularly important in the sector of sealants and adhesives. However, single-component hardening systems can potentially also be of use in fields that extend beyond these in the medical sector, e.g. in the dental sector, for coatings such as lacquers, or for reactive resins, e.g. roadmarkings or industrial floorcoverings.

There are many industrial methods for providing single-component systems. Firstly, the hardening mechanism can be initiated by a component provided by subsequent diffusion, preferably from the environment, an example being oxygen or atmospheric moisture. However, moisture-curing systems, mostly isocyanate-based or silyl-based, are not suitable for every application. By way of example, moisture-curing systems are not very suitable for very thick layers or applications in wet areas. Systems of this type moreover cure only very slowly, often requiring weeks for complete hardening. In contrast, by way of example, roadmarkings require rapid hardening.

A second industrial solution for providing single-component coating systems (hereinafter abbreviated to 1C systems) with good shelf life is encapsulation of a reaction component, e.g. a crosslinking agent, a catalyst, an accelerator, or an initiator.

Fast hardening mechanisms of this type play a major role in particular for reactive resins. Reactive resins mostly cure by way of free-radical reaction mechanisms. The initiator system here is in most cases composed of a free-radical chain initiator, mostly made of a peroxide or a redox system, and of an accelerator, mostly amines. Both components of the system can be encapsulated per se. However, a problem in the prior art is the release mechanism by which the capsules are ruptured, dissolved, or otherwise opened.

### PRIOR ART

There are systems that have been known for quite some time which release active ingredients or reaction components, comprising organic or inorganic porous matrices from which the active ingredient is slowly released. This type of system has the disadvantage that the release of the active ingredient is extended over a prolonged period, but there is no way of controlling the start of the release. As an alternative to porous matrices, it is also possible to use core-shell particles where the active ingredient is present in the core and the shell has sufficient permeability for said active ingredient to ensure controlled release over a prolonged period. An example of peroxide-containing particles produced by means of absorption is found in WO 00 15694. An alternative material for absorption is quartz particles as described in WO 94 21960. However, these particles are again likely to give only very restricted shelf life in a 1C system.

In contrast to this, in encapsulated systems it is possible to control the time of release. Core-shell particles are mostly involved here, where the shell of these is impermeable to the active ingredient and the particles have to be opened to release the active ingredient. There are a number of known release mechanisms. These can be based either on external energy input or on alteration of a chemical formulation parameter, such as moisture content or pH. However, a disadvantage of release caused by introduction of water or of solvent is that these methods either function very slowly or require an addition. In the latter case, the system would have the features and disadvantages of a 2-component system. In the former case, release would be too slow for applications such as roadmarking.

There are now established systems in which the opening mechanism is based on pressure, or on introduction of mechanical energy, for example through shear. To this end, various coatings have been described for encapsulating reactive components such as initiators. These systems are based on organic, thick-layer coatings. A disadvantage of these systems of the prior art is mostly that the shells lack shear resistance. It is therefore mostly difficult to incorporate these core-shell particles into a 1C system since the shear energy arising in the mixing process here is too high for the relatively unstable shells. This effect is mostly countered by producing particles of diameter smaller than 500 μm. However, small particles have the disadvantage of requiring a relatively large amount of shell material, or a significantly greater number of particles, for a relatively small amount of fill material, such as a peroxide dispersion. The residues of the particles remain in the formulation applied, where they can cause disadvantageous effects, such as haze, phase separation, loss of adhesion, softness or relatively low Shore hardness values, or coagulation. The objective for this type of 1C system should therefore be to minimize content of the shell material. Relatively small particles are also more difficult to rupture than relatively large particles. This can lead to incomplete provision of the reactive component and can sometimes lead to a requirement for a further increase in content of the formulation. Examples of these organic shell materials for encapsulating reactive components, or solutions or dispersions comprising these, are mainly polymers obtained from natural sources, e.g. gelatin, carrageenan, gum Arabic, or xanthan, or chemically modified materials from this type of source, e.g. methylcellulose or gelatin polysulfate. Lists and encapsulation examples using these materials for synthesizing core-shell particles with maximum size 500 μm can be found in GB 1,117,178, WO 98 2865, U.S. Pat. No. 4,808,639, DE 27 10 548, and DE 25 36 319. Combinations of various materials, such as gelatin and gum Arabic, have also been described (see McFarland et al., Polymer Preprints, 2004, 45(1), pp. 1 ff.); Bounds et al., Polymer Preprints, 2008, 49(1), pp. 777 ff.).

A particular case is provided by biocompatible capsule materials, for example for dental applications. One example of these has shells made of polyethyl methacrylate (Fuchigami et al., Dental Material Journal, 2008, 27(1), pp. 35-48). However, the person skilled in the art can easily see that these core-shell particles are difficult to open, and have to be extremely small for this type of application restricted to small application areas or application volumes.

An alternative here is provided by synthetic encapsulation resins, such as polyethylene-maleic anhydride, epoxy resins, or polyvinyl alcohol-resorcinol resin, and these can be found in the same publications. Phenol-formaldehyde resins (U.S. Pat. No. 5,084,494) and other formaldehyde-based resins (EP 0 785 243) have been particularly intensively studied. However again the only capsules described using these materials have an overall diameter of at most 200 μm or at most 100 μm. It is also possible to use peroxide solutions enclosed by metallic soaps of C₄-C₃₀-carboxylic acids, as in WO 03 082734. However, the person skilled in the art can easily see that these capsules are highly unstable, and accordingly they are also described only with a maximum size of 500 μm.

NL 6414477 describes the construction of a shell by means of polycondensation to give polyesters or polyamides. However, these capsules are either too permeable for the material enclosed within the core or too difficult to open. The encapsulation mechanism using condensation polymerization in the presence of the reactive substance to be encapsulated is moreover a complicated process which mostly does not proceed to completion.

WO 94 21960 describes a 1C system based on polyester for roadmarkings. However, this involves what really amounts to a 2C system, where beads which bear the hardening catalyst on the surface are added to the resin syrup during application. The person skilled in the art can easily see that this is not actually a 1C system with good shelf life. The beads are composed of sodium salts of organic acids such as naphthalenesulfonic acid or polycarboxylic acids, or are composed of quartz. U.S. Pat. No. 4,917,816 describes particles of this type of size about 10 μm for other applications.

### OBJECT

It was an object of the present invention to provide a novel single-component coating system—hereinafter abbreviated to 1C system—in particular suitable for roadmarking on various substrates, which have good shelf life and lack at least some of the disadvantages of the 1C systems of the prior art, or have these only to a reduced extent.

A particular object consisted in providing a 1C system which can be activated through a mechanism of maximum simplicity.

Another object consisted in providing 1C systems comprising core-shell particles, characterized in that only a relatively small amount of shell material is required in the formulation, in comparison with the prior art, and the core-shell particles can be activated in such a way that the reactive component present within the core is almost completely released within a very short time for the hardening of the 1C system.

Another object was to provide, for use as coating, a 1C system which is intended to be versatile and capable of flexible formulation, and to have relatively good shelf life.

Other objects not explicitly mentioned will be apparent from the entire description, claims and examples below.

### Achievement of Object

The objects are achieved by providing a novel 1C system which comprises core-shell particles. In particular, the 1C system involves a formulation comprising (meth)acrylates.

The term (meth)acrylate here means either methacrylate, e.g. methyl methacrylate, ethyl methacrylate, etc. or acrylate, e.g. methyl acrylate, ethyl acrylate, etc., and also mixtures of these two.

The core-shell particles comprise a reactive component within the core. This can take the form of pure substance, solution, or dispersion. It is preferable that it involves a solution or a dispersion of a reactive component in an organic solvent, oil, or in a plasticizer. The shells of the core-shell particles are moreover composed of an inorganic material, preferably of a silicate, particularly preferably of sodium silicate, i.e. of waterglass.

Another distinguishing feature of the core-shell particles is that they have a particle size of at least 100 μm, preferably of at least 200 μm, in particular embodiments at least 500 μm. The maximum particle size is 3 mm, preferably 1.5 mm, and particularly preferably 800 μm. Surprisingly, it has been found that these particles, which are large in comparison with those of the prior art, on the one hand provide particularly good shelf life but on the other hand are capable of a rapid opening process which proceeds almost to completion.

The shell makes up from 40% by weight to 75% by weight of the mass of the filled core-shell particle, preferably from 60% by weight to 70% by weight.

In this specification, the expression particle size means the actual average primary particle size. Since formation of conglomerates has been excluded, the average primary particle size is the same as the actual particle size. The particle size moreover corresponds approximately to the diameter of an approximately spherical particle. In the case of non-spherical particles, the average diameter is determined as average value from the shortest and longest diameter. In this context, diameter means a distance along a line from one point on the periphery of the particle to another. This line must also pass through the center of the particle. The person skilled in the art can determine the particle size by using, for example, a microscope, such as a phase-contrast microscope, or in particular an electron microscope (TEM), or by microtomography, e.g. by measuring a representative number of particles (e.g. 50 or >50 particles), using an image evaluation method.

In the ideal case, the core-shell particles are almost spherical. However, the particles can also be bar-, droplet-, plate-, or cup-shaped. The surfaces of the particles are generally rounded surfaces, but they can also exhibit other types of (inter)growth. As is known, an aspect ratio can be stated to serve as a measure of approximation of the geometry to the spherical shape. The maximum aspect ratio arising here deviates by at most 50% from the average aspect ratio.

The invention is particularly suitable for producing core-shell particles with an average aspect ratio of at most 3, preferably at most 2, particularly preferably at most 1.5. The expression maximum aspect ratio of the primary particles means the maximum ratio that can be calculated from two of the three dimensions length, width, and height. The ratio calculated here is always that of the largest dimension to the smallest of the other two dimensions.

The composition of the core-shell particles can also very occasionally take the form of secondary particles composed of up to 10 primary particles. The maximum size of these secondary particles depends on that of the individual primary particles present and is 3 mm, preferably 1.5 mm, and particularly preferably 800 μm.

The reactive component present within the core of the core-shell particles involves a compound for hardening the coating system. It preferably involves an initiator, catalyst, or accelerator, and particularly preferably involves an initiator for a free-radical polymerization reaction, preferably an organic peroxide.

The novel 1C system comprising core-shell particles has the advantage of good shelf life. In the invention, a 1C system is a formulation which once formulated can be stored for a particular period and then without further formulation or addition of any additional component can be applied and hardened. This requires activation of the system. Here, this involves the controlled release of a reactive component during application of the system. A first advantage of the 1C system of the invention is good shelf life. The 1C system of the invention has a shelf life of at least three, preferably at least six, months, and can then be used directly without addition of other components.

Another advantage of the system of the invention is that, in comparison with the prior art, the release of the reactive component from the core-shell particles can be achieved very rapidly and almost to completion during application as coating. The release of the reactive component is achieved by means of rupture of the shells through exposure to pressure or to any other form of mechanical energy. At least 80%, preferably at least 90%, particularly preferably at least 95%, of the reactive component is released here within 2 min, particularly preferably within 1 min. Hardening of the roadmarking to the extent that traffic can pass over the same is achieved within a period of 12 min from the juncture of rupture of the shell, preferably within a period of 8 min. In the particular embodiment of a rapid-hardening roadmarking, traffic can again pass over the same within a period of 2 min, preferably within a period of 1 min. This interval comprises the application procedure after the rupture of the shells, and any steps following this, for example the embedding of glass beads.

One particular aspect of the invention in this connection proves to be that the core-shell particles are markedly larger than in the prior art. This size provides more complete and faster destruction of the shells during application while also, by virtue of greater shell thickness, providing improved shelf life not only in respect of diffusion through the shell but also in respect of premature destruction of the particles through temperature changes or introduction of relatively small amounts of mechanical energy, e.g. shear energy during formulation or transport, or during any possible redispersion or mixing process.

A shell-destruction mechanism based on introduction of mechanical energy is preferred in respect of shelf life and also in respect of speed and/or completeness of destruction, over opening mechanisms based on diffusion, chemical reaction, change of pH or of polarity, or on radiation, and is preferred especially in respect of shelf life over mechanisms based on introduction of heat. This type of mechanism using introduction of mechanical energy can therefore be used with particular ease and advantage.

The particular size of the core-shell particles used in the invention also provides particles that are stable with respect to formulation and transport and to introduction of other relatively small amounts of energy, but which comprise only a relatively small proportion of the shell material. Smaller particles of the prior art either have only very low shell thicknesses or are naturally composed of very large proportions, or more precisely predominant proportions, of shell material. The core-shell particles used in the invention are composed of at most 75% by weight, preferably at most 70% by weight, of shell material. By virtue of this combination, advantageous compared to the prior art, of shell thickness and shelf life associated therewith and relatively high active-ingredient content, the amount of shell material to be found in the coating after application is only relatively small. The residual shell material can have attendant disadvantageous effects in some applications, an example being reduced adhesion, reduced cohesion, or haze.

The core-shell particles comprise, based on the total mass of the particle, at least 10% by weight, preferably at least 20% by weight, particularly preferably at least 30% by weight, of reactive component.

Another effect of this advantageous structure of the particles is that the coating system has to comprise only relatively small amounts of core-shell particles, more precisely at most 15% by weight, preferably at most 10% by weight, particularly preferably at most 5% by weight.

It has been shown that the amount of core-shell particles necessary in order that adequate hardening can be ensured at a hardening rate conventional in applications is at least 1% by weight, preferably at least 2% by weight.

As previously stated, the encapsulated reactive component involves a substance which is needed for hardening of the coating formulation. This can by way of example involve an aqueous solution of a catalyst for silyl- or urethane-based moisture-crosslinking systems. Examples of catalysts for controlling the curing rate of silyl systems are boron trifluoride complexes, and also iron carboxylates, titanium carboxylates, or tin carboxylates.

Systems that harden by a free-radical route, for example resins based on (meth)acrylate, require a source of free radicals. This can by way of example involve UV initiators, such as benzophenone, which, after release, are exposed to natural light or to radiation from a source specifically used.

The reactive component can also involve a thermally activatable polymerization initiator. Polymerization initiators used are in particular peroxides and azo compounds. It can sometimes be advantageous to use a mixture of various initiators. It is preferable to use, as free-radical initiator, azo compounds, such as azobisisobutyronitrile, 1,1′-azobis(cyclohexanecarbonitrile) (WAKO® V40), or 2-(carbamoylazo)isobutyronitrile (WAKO® V30), or peresters, such as tert-butyl peroctoate, di(tert-butyl) peroxide (DTBP), di(tert-amyl) peroxide (DTAP), tert-butylperoxy 2-ethylhexyl carbonate (TBPEHC), and other peroxides that decompose at high temperature. Further examples of suitable initiators are dioctanoyl peroxide, didecanoyl peroxide, dilauroyl peroxide, dibenzoyl peroxide, di(monochlorobenzoyl) peroxide, di(dichlorobenzoyl) peroxide, p-di(ethylbenzoyl) peroxide, tert-butyl perbenzoate, or azobis(2,4-dimethyl)valeronitrile. For reactive resins for use by way of example for roadmarkings, particular preference is given to dilauroyl peroxide or dibenzoyl peroxide.

The initiator system can also involve a redox initiator system, one component of which is present in encapsulated form and the other component of which is present separately therefrom likewise in encapsulated form, or is preferably present in solution in the coating system. These systems can by way of example involve a combination of hydroperoxides, such as cumene hydroperoxide, or ketone peroxides, and activators, for example acidic vanadium phosphates.

One particular embodiment of a redox initiator system for reactive resins such as those used by way of example for roadmarkings is a combination of peroxides, for example dilauroyl peroxide or dibenzoyl peroxide, and accelerators, in particular amines. Examples that may be mentioned of said amines are tertiary aromatically substituted amines, such as in particular N,N-dimethyl-p-toluidine, N,N-bis(2-hydroxyethyl)-p-toluidine, or N,N-bis(2-hydroxypropyl)-p-toluidine.

Another advantage of the present invention is that the filled core-shell particles are self-sealing in a reactive resin. Hair cracks or microcracks are sealed by polymerization of monomer that penetrates into the particles, without any risk that this local reaction might propagate initiation into the resin. This effect is present irrespective of whether the encapsulated reactive component involves the initiator or involves an accelerator.

In particular for the use in roadmarkings, preference is given to a redox initiator system based on a peroxide and on an accelerator. Very particular preference is given to a coating system for roadmarking in which the peroxide has been encapsulated as solution or dispersion within the core-shell particles.

The reactive component preferably takes the form of solution or dispersion in a solvent, oil, or plasticizer. Solvents that can be used are any of the organic liquids which are immiscible with water or have only poor miscibility therewith, and which are not reactive toward the reactive component. Particularly relevant materials here are aromatics, such as toluene or xylene; or solvent mixtures comprising aromatics, for example naphtha; acetates, such as ethyl, propyl, or butyl acetate; ketones, such as acetone or methyl ethyl ketone (MEK); or aliphatics, such as hexane or heptane. It is also possible to use mixtures of various solvents.

Plasticizers that can be used are phthalates, fatty acid esters, or short-chain polyethers. Oils are in particular Drakesol 260 AT, Polyoel 130, and Degaroute W3, particularly preferably Dagaroute W3. In order to ensure that the oil comprises no residual water, it can be dried prior to use, e.g. by thermal treatment in a drying oven. The hardening of, for example, waterglass proceeds more rapidly and more effectively when the included oil is anhydrous.

The concentration of the reactive component in the solution or dispersion can be selected freely at any level up to 100%, and is not subject to any further restriction.

Dispersions of a peroxide, such as dibenzoyl peroxide, in Degaroute W3 with peroxide concentration from 10% by weight to 80% by weight, preferably from 20% by weight to 70% by weight, and particularly preferably from 40% by weight to 60% by weight, have proven particularly advantageous for the use by way of example as system for roadmarking. The peroxide used here can already comprise small amounts of a phlegmatizer or water, e.g. 10% by weight.

The monomers present in the 1C system involve compounds selected from the group of the (meth)acrylates, such as alkyl (meth)acrylates of straight-chain, branched, or cycloaliphatic alcohols having from 1 to 40 carbon atoms, e.g. methyl (meth)acrylate, ethyl (meth)acrylate, n-butyl (meth) acrylate, 2-ethylhexyl (meth) acrylate, stearyl (meth)acrylate, lauryl (meth)acrylate; aryl (meth)acrylates, such as benzyl (meth)acrylate; mono(meth)acrylates of ethers, of polyethylene glycols, of polypropylene glycols, or mixtures of these having from 5 to 80 carbon atoms, for example tetrahydrofurfuryl (meth)acrylate, methoxy (m)ethoxyethyl (meth)acrylate, benzyloxy methyl (meth)acrylate, 1-ethoxybutyl (meth) acrylate, 1-ethoxyethyl (meth) acrylate, ethoxymethyl (meth)acrylate, poly(ethyleneglycol) methylether (meth)acrylate, and poly(propyleneglycol) methylether (meth)acrylate.

Other suitable constituents of monomer mixtures are additional monomers having a further functional group, for example α,β-unsaturated mono- or dicarboxylic acids, such as acrylic acid, methacrylic acid, or itaconic acid; esters of acrylic acid or methacrylic acid with dihydric alcohols, for example hydroxyethyl (meth)acrylate or hydroxypropyl (meth)acrylate; acrylamide or methacrylamide; or dimethylaminoethyl (meth)acrylate. Examples of other suitable constituents of monomer mixtures are glycidyl (meth)acrylate and silyl-functional (meth)acrylates.

The monomer mixtures can also comprise, alongside the (meth)acrylates described above, other unsaturated monomers which are copolymerizable with the abovementioned (meth)acrylates by means of free-radical polymerization. Among these are inter alia 1-alkenes and styrenes. Specific selection of the proportion and constitution of the poly(meth)acrylate is advantageously made with a view to the desired technical function.

Resins for roadmarking, this being a preferred use of the 1C systems of the invention, without any resultant restriction of the present invention to said use, can comprise further components alongside the starter system and the monomers. Specifically, the following components can also be present:

In what are known as MO-PO systems, there are also polymers present, preferably polyesters or poly(meth)acrylates, alongside the monomers listed. These are used in order to improve polymerization properties, mechanical properties, adhesion to the substrate, and also the optical properties required from the resins. The polymer content of the resin here is from 15% by weight to 50% by weight, preferably from 20% by weight to 35% by weight. Not only the polyesters but also the poly(meth)acrylates can have additional functional groups in order to promote adhesion or for copolymerization in the crosslinking reaction, for example taking the form of double bonds.

The monomers of which said poly(meth)acrylates are composed are generally the some as those previously listed in relation to the monomers in the resin system. They can be obtained by solution polymerization, emulsion polymerization, suspension polymerization, bulk polymerization, or precipitation polymerization, and they are added in the form of pure material to the system. Said polyesters are obtained in bulk via polycondensation or ring-opening polymerization, and are composed of the units known for these uses.

Other auxiliaries and additives that can be used are chain-transfer agents, plasticizers, crosslinking agents, stabilizers, inhibitors, waxes, oils and/or antifoams. Chain-transfer agents that can be used are any of the compounds known from free-radical polymerization. It is preferable to use mercaptans, such as n-dodecyl mercaptan. Examples of other suitable auxiliaries and additives are paraffins and crosslinking agents, in particular polyfunctional methacrylates, such as butanediol 1,4-di(meth)acrylate, tetraethylene glycol di(meth)acrylate, triethylene glycol di(meth)acrylate, trimethylolpropane tri(meth)acrylate, or allyl (meth) acrylate.

Plasticizers used are preferably esters, polyols, oils, or low-molecular-weight polyethers, or phthalates. From the group of the stabilizers and inhibitors, it is preferable to use substituted phenols, hydroquinone derivatives, phosphines, and phosphites.

Antifoams are preferably selected from the group of the alcohols, hydrocarbons, paraffin-based mineral oils, glycol derivatives, derivatives of glycolic esters, and acetic esters, and polysiloxanes.

Other materials that can be added to the 1C systems are dyes, glass beads, tine and coarse fillers, wetting agents, dispersing agents, and flow-control agents, UV stabilizers, and rheology additives.

Auxiliaries and additives preferably added when the 1C systems are used in the trafficway marking or surface-marking sector are dyes. Particular preference is given to white, red, blue, green, and yellow inorganic pigments, and titanium dioxide is particularly preferred.

Glass beads are preferably used as reflectors in formulations for trafficway marking and surface marking. The diameters of the commercially available glass beads used are from 10 μm to 2000 μm, preferably from 50 μm to 800 μm. The glass beads can also be silanized for easier use and better adhesion.

Fine fillers and coarse fillers can also be added to the formulation. These materials also have antiskid properties and are therefore in particular used in floorcoatings. Fine fillers used are those from the group of the calcium carbonates, barium sulfates, powdered and other quartzes, precipitated and fused silicas, pigments, and cristobalites. Coarse fillers used are quartzes, cristobalites, corundums, and aluminum silicates.

Wetting agents, dispersing agents, and flow-control agents used are preferably selected from the group of the alcohols, hydrocarbons, glycol derivatives, derivatives of glycolic esters, and acetic esters, and polysiloxanes, polyethers, polysiloxanes, polycarboxylic acids, and saturated and unsaturated polycarboxylic aminoamides.

It is equally possible to use conventional UV stabilizers. The UV stabilizers are preferably selected from the group of the benzophenone derivatives, benzotriazole derivatives, thioxanthonate derivatives, piperidinolcarboxylic ester derivatives, or cinnamic ester derivatives. Rheology additives preferably used are polyhydroxycarboxamides, urea derivatives, salts of unsaturated carboxylic esters, alkylammonium salts of acidic phosphoric acid derivatives, ketoximes, amine salts of p-toluenesulfonic acid, amine salts of sulfonic acid derivatives, or else aqueous or organic solutions or mixtures of the compounds. Rheology additives based on fumed or precipitated, optionally also silanized, silicas with BET surface area from 100 to 800 m²/g have been found to be particularly suitable.

The 1C systems of the invention using core-shell particles comprising a reactive component can be used in the form of resins, also termed reactive resins, for trafficway markings, or floorcoatings, for example on asphalt, concrete, or clay-based products, or else on old coatings or markings, for renovation. The hardening of the resins and formulations to the extent that traffic can pass over the same is achieved by free-radical polymerization within 12 min after release of the reactive component, preferably within 8 minutes. Other application sectors for reactive resins are casting compositions and moldings, e.g. for medical uses, examples being prostheses.

A major advantage of the 1C systems of the invention, comprising at least one encapsulated reactive component and monomers based on (meth)acrylate, is provided by the large amount of freedom with respect to formulation. It is possible to make a relatively free selection from all of the other components which have been listed by way of example for use as roadmarking. It is possible, for example, to start from known formulations for 2C coating systems when optimizing the 1C system for the substrate to be coated. It is thus possible to use the systems of the invention in an appropriately matched formulation for marking of old coatings, concrete, asphalt, clay-based products, tar, or other road surfaces. Specific adjustment of the systems for the respective substrate is necessary because the adhesion properties of the surfaces can differ very greatly.

When the 1C coating systems of the invention, which must comprise an encapsulated reactive component and a (meth)acrylate-based monomer system, are formulated appropriately, they can moreover be used for quite different uses and surfaces, for example metals, plastics, glass, ceramic, organic tissue, or wood. This gives a very wide range of possible further uses, e.g. in primers, lacquers, paints, adhesives, sealants, or for coating food, feed, or pharmaceutical products, or in dental materials or cosmetics. This list has no restrictive effect of any kind on the range of possible applications.

### EXAMPLES

### Production of Peroxide-Filled Core-Shell Particles

### Equipment

Rheometer: Haake RheoStress 600

Measurement system: plate (solvent trap)/cone, DC 60/2°

Material charged to specimen vessel: 5.9 mL sodium waterglass

Measurement temperature: 23.0° C.

Measurement: after 120 s at 500 revolutions per s

Frequency generator: Black Star 1325 and Jupiter 2000

Transformer: Heinzinger LNG 16-6 (or similar equipment)

Lamp: Drelloscop 2008

Pumps:
piston diaphragm pump+pulsation damper: LEWA EEC 40-13
gear pump: Gather CD 71K-2

Flow rate through pumps: for 350/500 μm nozzles
piston diaphragm pump+pulsation damper for waterglass: from 1.5-5 l/h
gear pump for initiator-oil suspension: from 1-2 l/h

### Pretreatment of Sodium Waterglass

1.3 L of commercially available sodium waterglass with 40% by weight solids content and dynamic viscosity 110 mPas is placed in a crystallization dish of diameter 19 cm. A magnetic stirrer with stirrer bar (length: 2 cm) is used to stir the material. Continuous and very vigorous stirring is required, so that the entire surface is kept in motion and a distinct vortex is formed. Viscosity is measured after 24 h in the rheometer, using the plate-and-cone system (DC 60/2°). Subsequent dilution or further dying may be carried out to give a solids content of 45% by weight. Dynamic viscosity rises here from 110 mPas to 310 mPas. The measurement is made by means of a rheometer.

### Production of Initiator Suspension

The suspension is produced by taking a 500 mL specimen bottle and filling it with Degaroute W3. 20% by weight of BPO 75 (benzoyl peroxide, 75% by weight in plasticizer, hereinafter abbreviated to BPO) is then carefully added stepwise. BPO remaining on the surface is incorporated into the body of the material by using a wooden spatula. For subsequent treatment, the suspension is treated with ultrasound in an ice bath (Ultraturrax). In each case, 1 min at stage one, 10 min at stage two and finally 3 min at stage three.

### Method—Production of Peroxide-Filled Particles

The sodium waterglass and the initiator suspension made of BPO and Degaroute W3 are placed in the corresponding feed vessel. The frequency generator and the light source are switched on, using a frequency of 16 kHz. The pumps for the sodium waterglass and the suspension are then switched on at similar times and a continuous flow is regulated. A 600 mL glass beaker with internal diameter 7.6 cm is used as collector vessel. This comprises 300 mL of the collector fluid composed of industrial ethanol and Tego Carbomer 340 FD in a ratio of 100:1.5. The collector fluid is stirred with the aid of a magnetic stirrer and stirrer bar, using a stirring rate of from 650 to 1200 revolutions per minute. The height from nozzle head to collector fluid in the dropwise addition process is 16 cm. The dropwise addition process is delayed until stirring has formed a vortex. Every 2-3 minutes, once the solution has become saturated, the glass beaker is replaced by another, comprising fresh collector fluid.

The collector solutions comprising particles are combined, and the particles are removed by filtration by way of a sieve with pore size smaller than 500 μm. The particles are then washed first with industrial ethanol and then with methyl methacrylate. Between the individual washes, the particles are in each case air-dried. Finally, 1% by weight of Aerosil 200 is admixed with the washed and dried particles.

TABLE 1
Microscopy

| | Nozzle | Diameter |
|---|---|---|
| Example | in μm | in μm |
| 1 | 350/500 | 1731 |
| 2 | 250/350 | 1718 |
| 3 | 150/350 | 845 |

The diameters were determined microscopically by using image analysis.

### Shelf Life Study

In each case, two 20 mL snap-lid glass containers are one-third filled with the core-shell particles from Examples 1 to 3, and the remaining space is filled with MMA. In each case one of the glass containers is stored at room temperature and the other at 40° C. After storage for each of one, two, and three weeks, the materials are monitored for any noticeable viscosity increase or indeed solidification of the MMA. The particles are also monitored for any change in size, shape, and color.

No polymerization or viscosity increase occurred in any of the examples within the three weeks. In a comparative test, the particles are ruptured by compression with a spatula and the time taken for the formulation to lose flowability is observed at room temperature. After from 7 to 8 minutes, all of the specimens had lost flowability, i.e. had hardened.

### Production of a Single-Component Reactive Resin

Dimensional stability and stability of adhesion were measured according to DAfStb-RiLi 01/DIN EN 1542 99 or according to DIN EN 1436.

Reactive Resin Examples

The components of the standard reactive resin from Table 2 are mixed with one another by stirring for 15 minutes. The composition is then further processed with the rheology additives and dispersion additives, by using a dispersion process for 5 minutes, to give a trafficway-marking paint. The titanium dioxide and the calcium carbonate are then respectively incorporated by dispersion for a further 10 minutes. Finally, the core-shell particles are incorporated by stirring for a further 2 minutes.

In comparative example Comp. ex. 1, initiator (BPC) and waterglass ground in a mortar are added separately instead of the core-shell particles.

TABLE 2

| | | | | |
|---|---|---|---|---|
| | | | | Comp. |
| Component | Starting material | Ex. 4 | Ex. 5 | ex. 1 |

Added materials and fillers

| | | | | |
|---|---|---|---|---|
| Rheology additive | Aerosil 200 | 0.25 g | 0.25 g | 0.25 g |
| Dispersion additive | TEGO Dispers 670 | 0.75 g | 0.75 g | 0.75 g |
| Rheology additive | Byk 410 | 0.25 g | 0.25 g | 0.25 g |
| Pigment | TR 92 titanium | 25 g | 25 g | 25 g |
| | dioxide | | | |
| Calcium carbonate | Omyacarb 5 GU | 136 g | 136 g | 136 g |

Standard reactive resin (with accelerator)

| | | | | |
|---|---|---|---|---|
| Methyl methacrylate | | 32 g | 32 g | 32 g |
| 2-Ethylhexyl acrylate | | 16 g | 16 g | 16 g |
| Hydroxypropyl | Monomers | 8 g | 8 g | 8 g |
| methacrylate | | | | |
| Triethylene glycol | | 8 g | 8 g | 8 g |
| dimethacrylate | | | | |
| Polymethyl | DEGALAN PM 685 | 24 g | 24 g | 24 g |
| methacrylates | | | | |
| Waxes, flow-control | | 1.3 g | 1.3 g | 1.3 g |
| agent | | | | |
| Stabilizer | Topanol O | 0.05 g | 0.05 g | 0.05 g |
| Accelerator | N,N-bis(2- | 1.3 g | 1.3 g | 1.3 g |
| | hydroxypropyl)-p- | | | |
| | toluidine | | | |

Core-shell particles

| Particles | Ex. 1 | Ex. 2 | |
|---|---|---|---|
| Amount used | 28 g | 35 g | — |
| of which waterglass | 22.4 g | 27.4 g | 23.4 g |
| inc. W3 | | | |
| of which benzoyl | 4.2 g | 5.3 g | 4.2 g |
| peroxide | | | |

The constitution and nature of the waxes and flow-control agents to be used are known to the person skilled in the art and do not affect the inventive aspect of the examples. The stated polymethyl methacrylates preferably involve suspension polymers with molecular weight (M_{w}, measured via gel permeation chromatography against a PMMA standard) from 40 000 to 80 000 and glass transition temperature T_{g} from 55° C. to 90° C., and the suspension polymer here can have small amounts of acid groups and/or of hydroxyl groups. The present examples used DEGALAN PM 685 from Evonik Rohm (M_{w} about 60 000; T_{g} about 64° C.). The selection of the polymers and the selection of the monomers have equally little restricting effect on the invention.

After three months, the flowability and shelf life of the compositions from Examples Ex. 4 and Ex. 5 are still unaltered. Nor is any settling of the core-shell particles observable. This proves that the trafficway-marking compositions comprising core-shell particles have good shelf life.

The composition from comparative example Comp. ex. 1 has hardened completely after 350 sec.

The effectiveness of the compositions from Ex. 4 and Ex. 5 is also studied. For this, in each case 20 g were ground in a mortar for 2 min and then spread as quickly as possible onto a film. This procedure is repeated respectively after one week and after three weeks. For results, see Table 3:

TABLE 3

| | | Curing time | Curing time |
|---|---|---|---|
| Specimen | Curing time | after 1 week | after 3 weeks |
| Ex. 4 | 380 sec | 390 sec | 370 sec |
| Ex. 5 | 360 sec | 360 sec | 370 sec |
| Comp. ex. 1 | 350 sec | — | — |

It has therefore also been shown that the hardening rate of the reactive resins comprising the core-shell particles of the invention is still the same after three weeks as directly after formulation.

1. A single-component coating system, comprising a core-shell particle and a (meth)acrylate, wherein:
the core-shell particle comprises a core and a shell;
the core-shell particle is spherical and its particle size is at least 100 μm and at most 3 mm;
the shell of the core-shell particle comprises an inorganic material; and
the core of the core-shell particle comprises a reactive component or a solution or a dispersion of the reactive component.

2. The coating system of claim 1, wherein the inorganic material comprises a silicate.

3. The coating system of claim 1, wherein the reactive component comprises a compound for hardening the coating system.

4. The coating system of claim 3, wherein the reactive component further comprises an initiator for a free-radical polymerization reaction.

5. The coating system of claim 1, wherein the core-shell particle comprises from 40% to 75% by weight of the shell.

6. The coating system of claim 1, wherein the coating system has a shelf life of at least three months, and then does not require additional components.

7. The coating system of claim 1, wherein the shell is ruptured by exposure to pressure or mechanical energy, and the reactive component is released.

8. The coating system of claim 7, wherein, after the exposure to pressure or mechanical energy, at least 80%, of the reactive component is released within 2 min.

9. The coating system of claim 1, wherein the coating system comprises at most 15% by weight of the core-shell particle.

10. A composition comprising the single-component coating system of claim 1, wherein the composition is at least one selected from the group consisting of a primer, a lacquer, a paint, an adhesive, a sealant, a food coating, a feed coating, a coating for a pharmaceutical product, a dental material, and a cosmetic.

11. A resin, comprising the single-component coating system of claim 1, wherein the resin is suitable for producing a casting composition, a floorcovering, a molding for a medical application, or a roadmarking.

12. A roadmarking, comprising a core-shell particle comprising a core, a shell, an amine, a (meth)acrylate, a polymer and a filler, dye, or both, wherein:
the core comprises an organic peroxide; and
the shell comprises waterglass.

13. The coating system of claim 1, wherein the inorganic material comprises waterglass.

14. The coating system of claim 3, wherein the reactive component further comprises an organic peroxide.

15. The coating system of claim 1, wherein the core-shell particle comprises from 60% to 70% by weight of the shell.

16. The coating system of claim 7, wherein, after the exposure to pressure or mechanical energy, at least 95% of the reactive component is released within 2 min.

17. The coating system of claim 7, wherein, after the exposure to pressure or mechanical energy, at least 80% of the reactive component is released within 1 min.

18. The coating system of claim 1, wherein the coating system comprises at most 10% by weight of the core-shell particle.

19. The coating system of claim 1, wherein the core of the core-shell particle comprises the reactive component not in a solution or dispersion.

20. The coating system of claim 1, wherein the core of the core-shell particle comprises a solution or a dispersion of the reactive component.
